(19) United States
(12) Patent Application Publication
(10) Pub. No.: US 2012/0302807 A1
(43) Pub. Date: Nov. 29, 2012

(54) METHANE REJECTION AND ETHYLENE RECOVERY

(75) Inventor: Wim Frans Elseviers, Des Plaines, IL (US)

(73) Assignee: UOP LLC, Des Plaines, IL (US)

(21) Appl. No.: 13/242,657

(22) Filed: Sep. 23, 2011

Related U.S. Application Data
(60) Provisional application No. 61/490,899, filed on May 27, 2011.

(51) Int. Cl. C07C 4/00

(52) U.S. Cl. 585/315

(57) ABSTRACT

A process for the production of an ethylene product stream from a reactor effluent stream includes passing the reactor effluent stream and an ethylene recycle stream to a deethanizer zone to provide a light hydrocarbon feedstream and a C₃+ stream. The light hydrocarbon stream goes to a demethanizer zone to provide a C₂ bottom stream and an overhead stream, then splitting the C₂ bottom stream into an ethane stream and an ethylene stream. The ethylene stream is divided into a first ethylene product stream and an ethylene co-feed stream, which is fed subsequently to the overhead stream to a pressure swing adsorption process producing an adsorber effluent stream during an adsorption step and a desorbed stream on desorption. The desorbed stream constitutes all or a portion of the ethylene recycle stream.

### CROSS REFERENCE TO RELATED APPLICATION

This application claims priority under 35 U.S.C. §119 from prior provisional application No. 61/490,899 which was filed on May 27, 2011, herein incorporated by reference.

### FIELD OF THE INVENTION

This invention relates to improving the purity of an ethylene stream for use in an oxygenate to olefins process and preferably a methanol to olefins process. During adsorption of ethylene, a portion of the impurities are also adsorbed. The invention relates more specifically to removing the adsorbed impurities from the adsorbent by displacing them with recycled ethylene during the adsorption stage.

### BACKGROUND OF THE INVENTION

Pressure swing adsorption (“PSA”) processes are well known for separating multi-component, gaseous feedstocks into a plurality of product streams, each of which is rich or lean in certain of the components. At least one product stream is rich in the target product while at least a second stream is rich in impurities or other waste materials. This process can be used, for example, to safely separate methane from a mixture of air and methane. It is also known to be useful for separation of hydrocarbons from mixtures that have been contaminated with small amounts of oxygen and/or air. Multi-stage pressure swing adsorption is also used to separate multiple product streams from the multi-component feedstock. A hydrocarbon stream rich in oxygenates is separable into a fuel gas stream, a stream rich in propylene, another stream rich in ethylene and a C₄+ hydrocarbon stream.

In each stage, an adsorber is filled with an adsorbent that is selected to adsorb the target product while most of the remaining feedstock is removed in an overhead stream. During an adsorbing phase, the adsorber is operated at high pressure and adsorbs the target product from a feedstock that enters the absorber at an inlet. A small portion of impurities in the remaining feedstock is also adsorbed while the remainder of the feedstock is removed from an outlet of the adsorber. When the capacity of the adsorbent is reached, flow of the feedstock to the adsorber is terminated.

The adsorber is then depressurized in an expansion phase while feedstock already present in the adsorber is removed through the outlet. As the pressure decreases, the target product expands and desorbs from the adsorbent, producing the product stream rich in the target product. The target product stream is withdrawn through the adsorber inlet and sent for further processing, if necessary. Following desorption, the adsorber is repressurized in preparation for receiving the multi-component, gaseous feedstock through the inlet.

In a continuous process, separation of each component utilizes multiple adsorbers to produce a continuous target product stream. While at least a first adsorber is actively adsorbing the target product, some of the remaining adsorbers are being regenerated. During regeneration, the adsorber is prepared to resume adsorption by depressurizing the adsorber, desorbing the target product and repressurizing the vessel. When flow of the feedstock is terminated to the first adsorber, at least a second adsorber can be pressurized and ready to accept the feedstock.

U.S. Pat. No. 4,498,910, herein incorporated by reference, describes a continuous process utilizing a plurality of interchangeable adsorbers. Each of the adsorbers cyclically performs the process steps of i) adsorbing the target product at elevated pressure; ii) withdrawing a gaseous phase at the outlet which is depleted in the target product; iii) withdrawing a stream of desorbed target product from the inlet and iv) pressurizing the adsorber to adsorption pressure. Ethylene was recovered from a feedstock of hydrocarbons and oxygen.

Ethylene and heavier components are separated from an oxygenate stream in U.S. Pat. No. 6,444,869. The PSA process is used to remove hydrogen, methane and ethylene from a demethanizer overhead stream. This ethylene is then continuously recombined with the oxygenate conversion process effluent stream to the compressor. Compressor effluent (which includes the recycled ethylene) then passes through steps of oxygenate removal, CO₂ removal, drying, compression and demethanization before entering an ethylene recovery zone.

### SUMMARY OF THE INVENTION

This invention relates to an improved process for recovering ethylene by enhanced rejection of methane, hydrogen, carbon oxides and nitrogen during the adsorption phase. A process is described for the production of an enriched ethylene product stream from a reactor effluent stream comprising hydrogen, nitrogen, carbon oxides, methane, ethylene, ethane, propylene, propane, and C₄+ olefins. The process includes passing the reactor effluent stream and an ethylene recycle stream at elevated pressure to a deethanizer zone to provide a light hydrocarbon feedstream having hydrogen, carbon oxides, nitrogen, methane, ethane and ethylene, and a deethanized C₃+ stream. The light hydrocarbon feedstream is passed to a demethanizer zone to provide a C₂ bottom stream rich in ethane and ethylene, and an overhead stream having hydrogen, carbon oxides, nitrogen, methane, and some residual ethane and ethylene. Next, the C₂ bottom stream is split in a C₂ splitter zone to produce an ethane stream and an ethylene stream, then dividing the ethylene stream into a first ethylene product stream and an ethylene co-feed stream. The overhead stream and the ethylene co-feed stream are subsequently fed as feed streams into an adsorption zone. The adsorption zone feed streams are each passed at effective adsorption conditions to an adsorption zone containing at least two adsorption beds, the overhead stream being passed through the adsorption beds prior to the ethylene co-feed stream, each of the adsorption beds containing a selective adsorbent to adsorb the ethylene to produce an adsorber effluent stream comprising hydrogen, carbon oxides, nitrogen and methane during the adsorption step, to produce a desorbed stream comprising ethylene on the desorption step and, immediately following adsorption, withdrawing a vent stream comprising hydrogen, carbon oxides, nitrogen and methane in a co-current depressurization step which is terminated prior to the breakthrough of ethylene. The desorbed stream is the ethylene recycle stream that is mixed with the reactor effluent stream to the de-ethanizer zone.

The process of this invention is advantageous as it produces a higher purity ethylene stream. In the adsorption zone, light gases such as hydrogen, carbon oxides, nitrogen and methane are quickly adsorbed, but are not adsorbed as strongly as ethylene. As the feedstock passes through the adsorbent bed, ethylene displaces the light gases as it is adsorbed more strongly. An ethylene front forms at the interface where hydrogen, carbon oxides, nitrogen and methane are being displaced and the ethylene is being adsorbed. Upon substitution of the overhead stream with the ethylene co-feed stream, the ethylene front moves nearer to the exit of the bed, while the light gases are pushed out of the adsorber, leaving it virtually full of ethylene. Thus, as the ethylene is desorbed, a higher-quality ethylene recycle stream is produced having fewer light gases present.

### BRIEF DESCRIPTION OF THE DRAWINGS

FIG. 1 is a schematic diagram of one embodiment of the method of this invention.

### DETAILED DESCRIPTION OF THE INVENTION

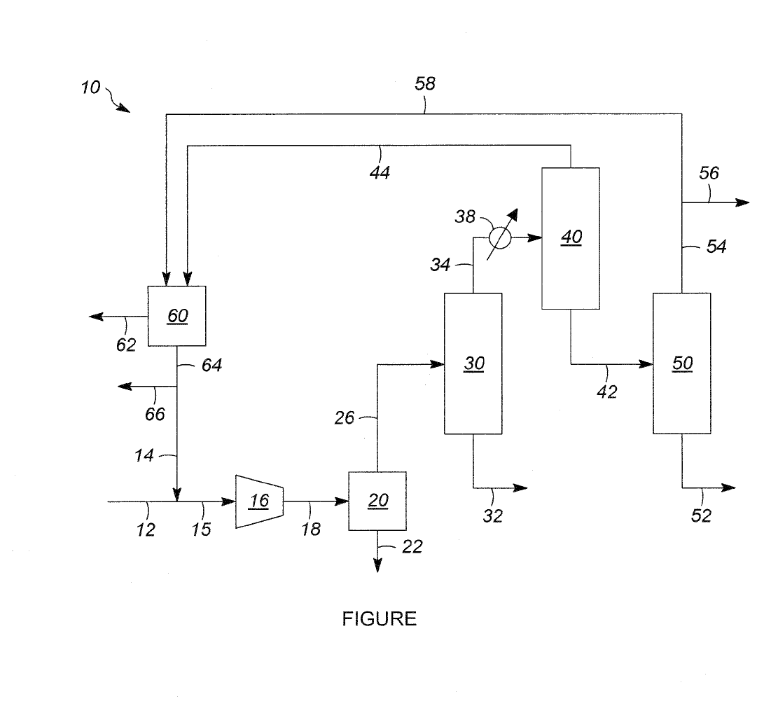

FIG. 1 illustrates various aspects of a process of recovering ethylene, generally 10, that embodies several aspects of the present invention. It is to be understood that no limitation to the scope of the claims which follow is intended by the following description. Those skilled in the art will recognize that these process flow diagrams have been simplified by the elimination of many necessary pieces of process equipment including some heat exchangers, process control systems, pumps, fractionation systems, etc. It may also be discerned that the process flow depicted in the figures may be modified in many aspects without departing from the basic overall concept of the invention.

Referring to FIG. 1, a reactor effluent stream 12 is shown coming from a reactor (not shown). In some embodiments, the reactor is an oxygenate conversion zone (not shown) and the reactor effluent stream 12 is an oxygenate conversion effluent stream. In the discussion below, the invention is described for use with an oxygenate conversion stream 12, but it not intended to limit the scope of the invention in any way.

The oxygenate conversion zone utilizes a hydrocarbon feedstock comprising one or more aliphatic hetero compounds that include alcohols, halides, mercaptans, sulfides, amines, ethers, carbonyl compounds or mixtures thereof, and converts them into a hydrocarbon product containing light olefinic products, such as C₂, C₃ and/or C₄ olefins. Typical processes for producing light olefins are described U.S. Pat. No. 4,499,327 B1 and U.S. Pat. No. 4,873,390 B1.

The oxygenate conversion effluent stream 12 includes hydrogen, carbon oxides (carbon monoxide and carbon dioxide), nitrogen, water, C₂ to C₄ hydrocarbons and oxygenates such as dimethyl ether (“DME”). This oxygenate conversion stream 12 is withdrawn from the oxygenate conversion zone, combined with an ethylene recycle stream 14 to make a combined feed 15 and passed to a multi-stage effluent compression zone 16 to raise the pressure of the oxygenate conversion effluent stream 12. This provides a compressed effluent stream 18 at a pressure of between about 1000 kPa (150 psia) and about 3000 kPa (450 psia). In some embodiments, the compression zone 16 raises the pressure of the compressed effluent stream 18 to a pressure between about 1750 kPa (250 psia) and about 2450 kPa (350 psia). Following the compression zone 16 is a flash drum 20 where a rapid decrease in pressure causes condensation of C₄ and heavier hydrocarbons 22.

A flash drum effluent stream 26, which is also the deethanizer feed stream, then passes to a series of fractionation zones to separate the individual olefins into higher purity products. As feedstock to a deethanizer zone 30, the flash drum effluent stream 26 is separated into a C₃+ fraction 32 and a deethanizer overhead stream 34 that is fed to a demethanizer zone 40. The deethanizer overhead stream 34 is a light hydrocarbon stream that includes hydrogen, nitrogen, methane and carbon oxides, but is particularly rich in ethylene and ethane. The deethanized C₃+ stream 32 includes propylene, propane and C₄+ olefins. Deethanizing conditions are maintained in the deethanizer zone, as will be understood by one of ordinary skill in the art. Deethanizer overhead stream 34 is conditioned by ethylene chiller 38 at a demethanizing temperature of between about −100° C. (−150° F.) and about −90° C. (−130° F.) to enter the demethanizer zone 40. The ethylene-depleted overhead stream 44 of the demethanizer is one of the feed gas streams to the separation zone 60, containing hydrogen, carbon oxides, nitrogen, methane, ethane and ethylene. The demethanizer bottom stream 42 is fed to the C₂ splitter zone 50 that produces an ethane stream 52 and an ethylene stream 54. Conditions on the demethanizer and the C₂ splitter are determined as dictated by the state of the art for these separations. A fraction of the ethylene stream 54 forms the ethylene co-feed stream 58 that is sent as a secondary feed gas stream to the separation zone 60, while the remainder of the ethylene stream 54 forms a first ethylene product stream 56.

The separation zone 60 utilizes any method whereby hydrogen, nitrogen, carbon oxides and methane compete with ethane or ethylene for adsorption onto the adsorbent. Examples of separation zones 60 are a solvent absorption zone, a temperature swing adsorption zone, a pressure swing adsorption zone, a vacuum swing adsorption zone, or a membrane separation zone and combinations thereof.

In an example of the present invention, the separation zone 60 includes a pressure swing adsorption process (“PSA”), having a series of multiple adsorber beds containing one or a combination of multiple adsorbents suitable for adsorbing the particular components to be adsorbed therein. These adsorbents include, but are not limited to, activated alumina, silica gel, activated carbon, zeolite molecular sieve type materials, or any combination thereof. The adsorbents are organized in any sequence as required by the adsorption process to adsorb ethylene and ethane. It is to be also understood that the term “counter-current” denotes that the direction of gas flow through the adsorption zone is counter-current with respect to the direction of PSA feed stream flow. Similarly, the term “co-current” denotes flow in the same direction as the PSA feed stream flow.

In the PSA process, PSA feed gas flows over the adsorbents and the more readily adsorbable components are adsorbed during the adsorption step. The remaining gas leaves the adsorber bed in the PSA overhead gas stream 62 that is rich in hydrogen, carbon oxides, nitrogen and methane. When the adsorbent has reached its adsorption capacity, it is regenerated to prevent a breakthrough of ethylene product into the PSA overhead gas stream 62. The first step of this regeneration is a series of one or more co-current depressurization steps that pressurizes any other bed at lower pressure with a pressure equalization step. Subsequently, or in parallel, co-current depressurization gas, or provide purge gas, is used to purge at least one other bed at low pressure. The flow direction is then reversed and the adsorber depressurizes counter-currently to the lowest pressure level in the system during a blowdown step. The blowdown step is followed by the purge step, during which provide purge gas from at least one other adsorber is used to regenerate the beds by efficiently desorbing the more readily adsorbable components. At the end of the purge step, the bed is considered sufficiently regenerated and can be pressurized with gas from other adsorbers on co-current depressurization, followed by or in parallel with a slipstream of the PSA feed or overhead gas produced during the adsorption step. A second ethylene product stream 64 is produced by desorption of the more readily adsorbable components during the blowdown and purge steps. It will be recycled fully or partially to the oxygenate conversion effluent stream 12. In case of partial recycle, stream 64 is split into an ethylene recycle export stream 66 and an ethylene recycle stream 14.

During the adsorption step, the capacity of the adsorber bed for the more readily adsorbable component reduces as more feed gas contacts the adsorbent. Several fronts of impurities move through the adsorber bed, with the lesser adsorbing components exiting first (e.g. hydrogen, nitrogen, methane). As adsorption progresses, the front of the more readily adsorbing ethylene also progresses toward the PSA overhead stream. Breakthrough of this front into the PSA overhead stream is undesirable as it results in a loss of ethylene production and recovery. In the prior art, a certain amount of light gases are present in the beds at the start of the regeneration phase. These light gases desorb during the blowdown and purge steps and are present in the second ethylene product stream 64 from the PSA.

However, in the present invention, preferably before the ethylene adsorption front has exited the adsorber bed, the flow of overhead feed stream 44 is discontinued and replaced by the ethylene co-feed stream 58. The fronts of the lighter gases from overhead feed stream 44 are pushed out while the ethylene front remains in the bed. As the concentration of ethylene adsorbed in the bed increases, the concentration of light gases adsorbed in the bed is reduced. The preferential adsorption of ethylene desorbs the lighter gases from the adsorbent bed while the ethylene front remains approximately at the same position in the beds without increasing the ethylene slip into the overhead gas, because ethylene will preferentially adsorb on the adsorbent space occupied by the adsorbed light gases in the absence of light gases in the secondary feed stream. Using this co-feed of more adsorbing ethylene co-feed stream 58 will thus result in a higher degree of removal of light gases for the same recovery of ethylene, or can achieve higher recoveries of ethylene for the same removal of light gases. Since less light gases remain adsorbed in the bed at the end of the adsorption step, the purity of the second ethylene product stream 64 produced by the PSA will be higher.

The point at which the transition is made from overhead feed stream 44 to ethylene co-feed stream 58 is determined during the unit design adsorption calculations and is set by the allowed ethylene losses. Since the ethylene front in the bed remains at approximately the same position, a measurement for this can be the concentration of ethylene in the overhead gas stream leaving from the bed. The selection of the exact value of the ethylene concentration allowed is determined by the adsorption calculations as a function of the allowed ethylene losses. A higher ethylene concentration in the overhead gas results in increased ethylene recovery losses but in smaller adsorption bed volumes. Thus, the selection of the transition point depends on the relative value of ethylene versus the investment cost of the PSA unit. Typically, the concentration of ethylene in the overhead gas is in the range 0.1% mole to 0.5% mole, though values as low as 0.01% mole and higher values up to 2% mole are suitable based on individual cases and economic situations.

There is no need for a continuous measurement of ethylene in order to control the adsorption. The actual loading of ethylene and light gases on the adsorber beds is only a function of the inlet gas composition, flow rate and the adsorption time. PSA processes are controlled on adsorption time, i.e. the allowed adsorption time is inversely proportional to the inlet feed flow rate, which loads the same amount of adsorbed components onto a fixed volume of adsorbent bed. Thus, controlling the adsorption time is the equivalent of controlling the concentration of ethylene in the overhead gas 62.

The adsorption temperature preferably ranges between about −18° C. and about 100° C. More preferably, the adsorption temperature ranges between about −18° C. and about 50° C. In most preferred embodiments, the adsorption temperature comprises less than about 49° C. It is believed that at adsorption temperatures above about 100° C., and especially at adsorption temperatures above about 50° C., silica gel becomes less selective for the adsorption of hydrogen and methane relative to C₂ hydrocarbons. This adsorption of ethylene over silica gel behaves in a manner which is opposite to the observed adsorption characteristics of ethylene over a zeolite adsorbent such as zeolite 4A.

The overhead PSA feed stream 44 includes from about 10 to about 60 mole % hydrogen, from about 5 to 50 mole % methane and from about 10 to 60 mole % ethylene and heavier components. There can be traces (ppm level to 1%) of carbon oxides and between 1 and 60 mole % nitrogen during certain operating conditions. In the separation zone 60, the more readily adsorbable components are adsorbed at an adsorption pressure and adsorption temperature and the less readily adsorbable components are passed through the adsorption zone. The adsorption zone pressure ranges from about 350 kPa to about 3.5 MPa (about 50 to about 500 psia), but can go as high as 8.3 MPa (about 1200 psia). The adsorption zone temperature is any temperature effective to adsorb the more readily adsorbable components in the feed stream, and preferably from about −18° C. to less than about 50° C. (about 0° to about 120° F.).

Following the adsorption and co-feed steps, the bed undergoes a step wherein the bed is allowed to depressurize co-currently to a fixed pressure in a direction co-current to the feeding step. By the use of this co-current depressurization step, the slippage of ethylene into the PSA overhead stream 62 is substantially reduced. The less readily adsorbable component that remains in the void spaces of the adsorbent bed ahead of the leading adsorption front can be essentially completely displaced from the bed and can enrich the more readily adsorbed component behind the adsorption front. This enables the more readily adsorbable component to be thereafter discharged from the feed end of the bed as a product of desired purity by counter-currently depressurizing the bed. The co-current depressurization step is optionally performed in conjunction with one or more co-current depressurization, or equalization, steps wherein the equalization gas is not vented, but passed to another bed. When a co-current depressurization step is used, it can be performed either before, simultaneously with, or subsequent to the vent step. The effluent stream from the co-current depressurization step, which is comprised primarily of less readily adsorbable components, can be used to partially repressurize or purge another adsorber bed. The combination of the desorption effluent resulting from the counter-current depressurization step and the purge step are combined to have ethylene and heavier product purity of from about 20 to about 95 mole %. In some embodiments, the first ethylene product stream 56 is recovered at a recovery of greater than about 99.5 mole % relative to the ethylene in the reactor effluent stream 12.

After the termination of the vent step and any desired co-current depressurization step(s), the adsorber bed is desorbed by reducing the pressure in a direction counter-current to the PSA feed direction to a desorption pressure that is preferably from about atmospheric pressure to about 350 kPa (about 50 psia). A portion of the desorption effluent stream recovered from the adsorption zone could be utilized as feed for the co-current displacement step following repressurization.

While a particular embodiment of the process for making an enriched ethylene product stream, it will be appreciated by those skilled in the art that changes and modifications may be made thereto without departing from the invention in its broader aspects and as set forth in the following claims.

What is claimed is:

1. A process for the production of an enriched ethylene product stream from a reactor effluent stream comprising hydrogen, methane, ethylene, ethane, propylene, propane, and C₄+ olefins, said process comprising:
a) passing the reactor effluent stream and an ethylene recycle stream at elevated pressure to a deethanizer zone to provide a light hydrocarbon feedstream comprising hydrogen, carbon oxides, nitrogen, methane, ethane and ethylene, and a deethanized C₃+ stream;
b) passing the light hydrocarbon feedstream to a demethanizer zone to provide a C₂ bottom stream comprising ethane and ethylene, and an overhead feed stream comprising hydrogen, carbon oxides, nitrogen, methane, ethane and ethylene;
c) passing the C₂ bottom stream to a C₂ splitter zone to produce an ethane stream and an ethylene stream;
d) dividing the ethylene stream into a first ethylene product stream and an ethylene co-feed stream;
e) passing the overhead feed stream and ethylene co-feed stream sequentially to an adsorption zone containing at least two adsorption beds, at effective adsorption conditions, each of said adsorption beds repeatedly going through (i) an adsorption phase wherein a mixture of selective adsorbents adsorb the ethylene to produce an adsorber overhead stream comprising hydrogen, carbon oxides, nitrogen and methane; (ii) one or more co-current depressurization phases which are terminated prior to the breakthrough of ethylene; (iii) a counter-current depressurization phase; (iv) a purge phase; and (v) a repressurization phase; wherein desorption of the desorbed ethylene recycle stream comprising ethylene is produced during the depressurization and purge phases; and
f) mixing at least a portion of the ethylene recycle stream with the reactor effluent stream, exporting the non-mixed portion into a separate export stream.

2. The process of claim 1 wherein the adsorption zone of said step e) is a temperature swing adsorption zone.

3. The process of claim 1 wherein one of said at least two adsorption beds is in the repressurization phase and is pressurized with vent gas from a second of said at least two adsorption beds that are in the co-current depressurization step.

4. The process of claim 1 wherein the selective adsorbent is selected from the group consisting of activated alumina, silica gel, activated carbon, zinc X zeolite, calcium Y zeolite, molecular sieves and mixtures thereof.

5. The process of claim 1 wherein the adsorption zone comprises a pressure swing adsorption process.

6. The process of claim 1 wherein the selective adsorbent comprises silica gel or mixtures of silica gel with activated carbon or activated alumina.

7. The process of claim 1 wherein the effective adsorption conditions include an adsorption temperature comprising less than about 49° C. (120° F.).

8. The process of claim 1 wherein the first ethylene product stream is recovered at a recovery of greater than about 99.5 mole % relative to the ethylene in the reactor effluent stream.

9. The process of claim 1 further comprising compressing the reactor effluent stream to an effective demethanizer pressure of between about 1000 kPa and about 3000 kPa prior to passing the reactor effluent stream to the demethanizer zone.

10. The process of claim 1 wherein the flow of the overhead feed stream is discontinued to the adsorber bed and the flow of the ethylene co-feed stream is initiated prior to breakthrough of an ethylene front.

11. The process of claim 1 wherein the reactor effluent stream comprises a reaction product of an oxygenate feedstock having been reacted at effective conversion conditions in the presence of a diluent in an oxygenate conversion reaction zone containing a small pore, non-zeolitic catalyst to convert essentially all of the oxygenate feedstock to produce the reactor effluent stream.

12. The process of claim 1 wherein the reactor effluent stream is compressed and flashed where a rapid decrease in pressure causes condensation of C₄ and heavier hydrocarbons.

13. The process of claim 1 wherein the reactor effluent stream is an oxygenate conversion effluent stream.
